(19) United States
(12) Patent Application Publication
(10) Pub. No.: US 2011/0312956 A1
(43) Pub. Date: Dec. 22, 2011

(54) ARYLINDENOPYRIMIDINES WITH REDUCED hERG CHANNEL BINDING

(76) Inventors: Paul JACKSON, New Hope, PA (US); Brian SHOOK, Gilbertsville, PA (US)

(21) Appl. No.: 12/816,680

(22) Filed: Jun. 16, 2010

(51) Int. Cl. A61K 31/5377; A61K 31/517; C07D 413/10; A61P 25/08; A61P 25/28; A61P 35/00; A61P 9/00; A61P 1/04; C07D 239/70; A61P 25/16

(52) U.S. Cl. 514/232.8; 544/249; 514/267; 544/115

(57) ABSTRACT

This invention provides novel arylindenopyrimidines of the Formula (I),

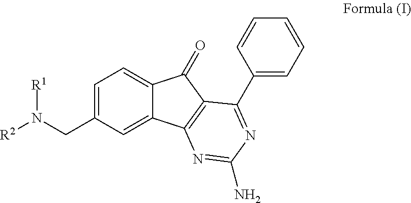

and pharmaceutical compositions comprising same, useful for treating disorders ameliorated by antagonizing adenosine A1 and/or A2a receptors. This invention also provides therapeutic and prophylactic methods using the instant pharmaceutical compositions.

### FIELD OF THE INVENTION

This invention relates generally to the fields of Adenosine A1, A2a receptor antagonists and their therapeutic and prophylactic uses. In particular, the present invention provides arylindenopyrimidines that are Adenosine A1, A2a receptor antagonists. More specifically, this invention provides certain arylindenopyrimidines with reduced hERG channel binding, as well as methods for the use of such arylindenopyrimidine compounds to therapeutically or prophylactically treat, prevent, reverse, arrest or inhibit conditions such as neurodegenerative and movement disorders while minimizing cardiac toxicity.

### DESCRIPTION OF THE RELATED ART

Adenosine is a purine nucleotide produced by all metabolically active cells within the body. Adenosine exerts its effects primarily via four subtypes of cell-surface receptors (A1, A2a, A2b and A3), which belong to the G protein coupled receptor superfamily (Stiles, G. L. Journal of Biological Chemistry, 1992, 267, 6451). A1 and A3 couple to inhibitory G protein, while A2a and A2b couple to stimulatory G protein. A2a receptors are mainly found in the brain, both in neurons and glial cells (highest level in the striatum and nucleus accumbens, moderate to high level in olfactory tubercle, hypothalamus, and hippocampus etc. regions) (Rosin, D. L.; Robeva, A.; Woodard, R. L.; Guyenet, P. G.; Linden, J. Journal of Comparative Neurology, 1998, 401, 163). In peripheral tissues, A2a receptors are found in platelets, neutrophils, vascular smooth muscle and endothelium (Gessi, S.; Varani, K.; Merighi, S.; Ongini, E.; Borea, P. A. British Journal of Pharmacology, 2000, 129, 2).

The striatum is the main brain region for the regulation of motor activity, particularly through its innervation from dopaminergic neurons originating in the substantia nigra. The striatum is the major target of the dopaminergic neuron degeneration in patients with Parkinson's Disease (PD). Within the striatum, A2a receptors are co-localized with dopamine D2 receptors, suggesting an important site for the integration of adenosine and dopamine signaling in the brain (Fink, J. S.; Weaver, D. R.; Rivkees, S. A.; Peterfreund, R. A.; Pollack, A. E.; Adler, E. M.; Reppert, S. M. Brain Research Molecular Brain Research, 1992, 14, 186).

Neurochemical studies have shown that activation of A2a receptors reduces the binding affinity of D2 agonist to their receptors. This D2R and A2aR receptor-receptor interaction has been demonstrated in striatal membrane preparations of rats (Ferre, S.; von Euler, G.; Johansson, B.; Fredholm, B. B.; Fuxe, K. Proceedings of the National Academy of Sciences of the United States of America, 1991, 88, 7238) as well as in fibroblast cell lines after transfection with A2aR and D2R cDNAs (Salim, H.; Ferre, S.; Dalal, A.; Peterfreund, R. A.; Fuxe, K.; Vincent, J. D.; Lledo, P. M. Journal of Neurochemistry, 2000, 74, 432). In vivo, pharmacological blockade of A2a receptors using A2a antagonist leads to beneficial effects in dopaminergic neurotoxin MPTP (1-methyl-4-pheny-1,2,3,6-tetrahydropyridine)-induced PD in various species, including mice, rats, and monkeys (Ikeda, K.; Kurokawa, M.; Aoyama, S.; Kuwana, Y. Journal of Neurochemistry, 2002, 80, 262). Furthermore, A2a knockout mice with genetic blockade of A2a function have been found to be less sensitive to motor impairment and neurochemical changes when they were exposed to neurotoxin MPTP (Chen, J. F.; Xu, K.; Petzer, J. P.; Staal, R.; Xu, Y. H.; Beilstein, M.; Sonsalla, P. K.; Castagnoli, K.; Castagnoli, N., Jr.; Schwarzschild, M. A. Journal of Neuroscience, 2001, 21, RC143).

The adenosine A1 receptor is also expressed in the striatum. Based on anatomical and in vivo microdialysis studies, A1 receptors appear to be localized pre-synaptically on DA axon terminals where they inhibit DA release. A1 receptor antagonists facilitate DA release in the striatum, and like A2a receptors, A1 receptors potentiate DA-mediated responses. Thus, inhibition of the A1 receptor will facilitate DA release, while inhibition of the A2a receptor will enhance post-synaptic responses to DA. Antagonism of both the A2a and A1 receptor would be synergistic in potentiating DA mediated responses.

Interestingly, the A1 receptor is also concentrated in neocortical and limbic system structures that are important for cognitive function and has been implicated in antidepressant action. Pharmacological inhibition of A1 receptors enhances neurotransmitter release in the hippocampus and enhances performance in animal models of learning and memory.

In humans, the adenosine receptor antagonist theophylline has been found to produce beneficial effects in PD patients (Mally, J.; Stone, T. W. Journal of the Neurological Sciences, 1995, 132, 129). Consistently, recent epidemiological study has shown that high caffeine consumption makes people less likely to develop PD (Ascherio, A.; Zhang, S. M.; Hernan, M. A.; Kawachi, I.; Colditz, G. A.; Speizer, F. E.; Willett, W. C. Annals of Neurology, 2001, 50, 56).

In summary, adenosine A2a receptor blockers can provide a new class of antiparkinsonian agents (Impagnatiello, F.; Bastia, E.; Ongini, E.; Monopoli, A. Emerging Therapeutic Targets, 2000, 4, 635). In addition, drugs that block both A2a and A1 adenosine receptors have enormous potential as therapeutic agents in a wide variety of disorders in humans

HERG Channel Binding

Human Ether-a-go-go-Related Gene (hERG) encodes potassium (K⁺) channels that are involved in such functions as maintenance of the resting membrane potential in all cell types and termination of action potentials in excitable cells. Hence, reduced hERG channel binding can have an important impact on conditions including cancer as well as cardiac, neurological, neuro-endocrinological, and gastro-intestinal diseases. For example, Curran et al. (Cell, 1995, 80, 795-803) report that regulating of hERG K⁺ channel is important in treating cardiac arrhythmia such as Long QT syndrome and ventricular fibrillation. Other researchers have reported that hERG K⁺ Channel can also be important in regulation of cancer cells, such as in colorectal cancer (Smith et al., J. Biol. Chem. 2002, 277, 18528-18534; Lastraioli et al., Cancer Res., 2004, 64, 606-611), as well as in treatment of neurological disorders such as episodic ataxia (Lehmann-Horn et al., Physiological Reviews, 1999, 79, No. 4, 1317-1372) and epilepsy (Danielsson et al., Epilepsy Res., 2003, 55(1-2):147-57).

One of the most important concerns in drug development is the possibility of a potential candidate having the potential for cardiac toxicity. Long QT syndrome (LQTS) is a disorder of ventricular repolarization that predisposes affected individuals to cardiac arrhythmia and sudden death. However, many drugs cause prolonged QT (acquired LQTS) and have potential for causing cardiac arrhythmias. Inherited LQTS is caused by mutations in K+ or Na+ ion channel genes or ankyrin-B (Keating, M. T., and Sanguinetti, M. C. (2001) Cell 104, 569-580; Mohler, P. J. et al. (2003) Nature 421, 634-639; and Fernandez D. et al. J. Biological Chemistry Vol. 279, No. 11, March 12, (2004) pp. 10120-10127)

Acquired LQTS is more common and can be induced as an unintended and extremely undesirable side effect of treatment with many structurally diverse medications. In the past few years, several commonly used drugs (e.g., terfenadine, cisapride, sertindole, thioridazine, grepafloxacin) were withdrawn from the market, or their approved use severely restricted when it was discovered that they caused cardiac arrhythmias or were associated with unexplained sudden death (Pearlstein, R. et al. (2003) J. Med. Chem. 46, 2017-2023).

The molecular basis of drug induced LQTS is blockage of human ether-a-go-go related gene (hERG) channels that conduct I_{Kr}, the rapid delayed rectifier K⁺ current important for repolarization of cardiac action potential. A reduction in I_{Kr} prolongs action potential duration of ventricular myocytes, lengthens the QT interval and increases dispersion as measured by electrocardiogram (ECG) recordings and increases the risk of Torsades de Pointes, a ventricular tachyarrhythmia that can degenerate into fibrillation and cause sudden death.

In a laboratory setting, it is possible to induce arrhythmia in animals with drugs that block voltage-gated K+ (Kv) channels other than hERG. However, in clinical practice, drug induced LQTS is always attributable to direct or indirect (via interference with metabolism of a co-administered medication) block of hERG channels (Redfern, W. S. et al. (2003) Cardiovasc. Res. 58, 32-45). This understanding has prompted intense efforts to quantify hERG channel activity of new chemical entities during an early stage of the drug development process (Fermini, B. and Fossa, A. A. (2003) Nat. Rev. Drug Discov. 2, 439-4477 and Fernandez D. et al. J. Biological Chemistry Vol. 279, No. 11, March 12, (2004) pp. 10120-10127).

Therefore in drug development it is extremely important to determine the potential of a candidate compound to block hERG channels. This property is not easily determined from the structure of the compound and closely related compounds may have vastly different potential to block hERG channels. Therefore, in drug development, there is an urgent need to develop compounds that have minimal ability to block hERG channels so as to produce drug candidates that will have minimal tendency to cause cardiac arrhythmias and/or sudden death in patients.

### SUMMARY OF THE INVENTION

This invention relates, in part, to compounds, methods and compositions useful for the treatment of A1 and/or A2a receptor-mediated conditions while at the same time producing minimal cardiac toxicity. Specifically, in part, the invention relates to compounds, methods and compositions to treat a condition that can be ameliorated by antagonizing Adenosine A1 and/or A2a receptors, which condition includes, but is not limited to, Parkinson's Disease, Huntington's Disease, Multiple System Atrophy, Corticobasal Degeneration, Alzheimer's Disease, and Senile Dementia.

One embodiment of such methods is a method to prevent the occurrence of a condition that can be ameliorated by antagonizing Adenosine A1 and/or A2a receptors while minimizing the risk of cardiac toxicity.

In addition, this invention relates, in part, to compounds, methods and compositions useful for the treatment, prevention, arrest, inhibition and/or reversal of an A1 and/or A2a receptor-mediated condition in a patient at risk for the development of such conditions while minimizing the risk of cardiac toxicity.

This invention is based, in part, on the previously unknown property of the arylindenopyrimidine compounds of the invention. These compounds are potent adenosine A1 and A2a receptor dual antagonists but a select group of these compounds have been unexpectedly found to have extremely low activity in blocking hERG channels even as compared to other structurally closely related compounds.

In various embodiments, the invention provides compounds, compositions and methods of treating, preventing, reversing, arresting or inhibiting a condition that can be ameliorated by antagonizing either or both adenosine A2a and A1 receptors in the subject while minimizing the risk of cardiac toxicity.

Accordingly, the present invention provides compounds, compositions and methods for treating, preventing, arresting, inhibiting and reversing an A1 and/or A2a receptor-mediated condition in a subject in need thereof while minimizing the risk of cardiac toxicity comprising administering to the subject a therapeutically effective amount of a composition that comprises at least one compound of Formula (I):

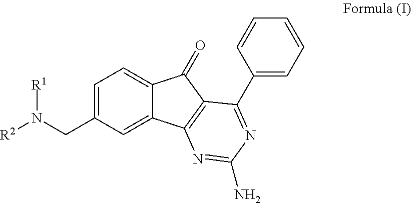

wherein
R¹ and R² are independently selected from H and optionally substituted C₁₋₃ alkyl,
or
R¹ and R² and the N atom they are attached to, together form optionally substituted heterocyclyl selected from; pyrrolidine, pyrrole, morpholine, piperidinone, pyrrolidone, piperidine, pyridine, dihydropyrrole and tetrahydropyridine, each heterocyclyl having 0-2 additional heteroatoms selected from O, S, and N;
or an optical isomer, enantiomer, diastereomer, racemate, or pharmaceutically acceptable salt thereof.
   The substituents present when R¹ and R² are a heterocycle are selected from; alkyl, hydroxy, carbonyl, carboxylic acid or esters and isosteres.
   The optional substitutions on the C₁₋₃ alkyl may be pyridine or furan.

The present invention also provides methods comprising administering to the subject a therapeutically effective amount of a composition that comprises at least one compound of Formula (I) wherein R¹ and R² and the N atom they are attached to together form optionally substituted heterocyclyl having 0-2 additional heteroatoms selected from O, S, and N.

In embodiments of the present invention, before prophylactic or therapeutic administration of the compounds or compositions of the invention to a subject, a determination will be made as to whether or not the subject suffers from one or more A1 and/or A2a receptor-mediated conditions, or is considered to be at a high risk for the development of such conditions.

In certain embodiments of the present invention, a therapeutically effective amount of a compound of Formula (I) is in a range of from about 0.05 mg/kg of body weight to about 50 mg/kg of body weight. The dosages, however, may be varied depending the individual characteristics and tolerances of the subject and the on the precise nature of the condition being treated.

In certain embodiments, a subject or patient in need of treatment may be a subject who has already shown the symptoms of an A1 and/or A2a receptor-mediated condition prior to the time of administration.

In another aspect, the subject or patient will be determined to be at risk for developing an A1 and/or A2a receptor-mediated condition.

Additional embodiments and advantages of the invention will become apparent from the detailed discussion, examples, and claims below.

### DETAILED DESCRIPTION OF THE INVENTION

This invention provides compounds, methods of treatment and compositions useful for the treatment of adenosine A1 and/or A2a receptor-mediated conditions while at the same time producing minimal cardiac toxicity.

### HERG Channel Binding Assay

The human ether-a-go-go related gene, hERG, expressed in the heart of human and other mammalian species encodes the pore-forming subunit of an ion channel called the rapidly activating delayed rectifier potassium current, Iₖᵣ. This ion channel is involved in repolarization of the cardiac action potential. Blocking the hERG K⁺ channels is an undesired property of pharmaceutics because inhibition of ion channels involved in cardiac repolarization can cause acquired long QT syndrome and may lead to a life-threatening form of ventricular arrhythmias called Torsade de Pointes. In susceptible individuals, syncope and sudden death can result from drug-induced block of this myocardial ion channel. Because the hERG K⁺ channel is a molecular target for QT prolongation by screening compounds for activity against this target at an early stage in drug discovery it is possible to select development candidates with reduced potential for undesirable side effects in humans, especially the possibility of causing cardiac arrhythmias.

A hERG binding assay is a primary screening method used to evaluate potential activity of a compound against the hERG potassium channel. The affinity of a compound for the hERG channel is measured in vitro by examining displacement of tritiated-[³H]astemizole, a high affinity hERG channel ligand. In this assay, membranes from HEK293 cells stably transfected with the human hERG channel cDNA are incubated with a range of concentrations of each test compound, vehicle (10% DMSO), or a positive control and [³H]astemizole.

After a 1-hr incubation, the membranes are harvested onto GF/B filters, the filters dried and the radioactivity is measured. For each test compound, percent inhibition of [³H]astemizole binding is calculated relative to control binding and IC₅₀ values determined by linear regression. Each assay includes a positive control (i.e., Cisapride) that is tested in parallel with test compounds. At present, there is no gold standard preclinical test available to predict QT prolongation in humans. Therefore to assess potential cardiotoxicity, cardiovascular effects are evaluated preclinically in several different tests including the hERG channel binding assay.

Co-pending U.S. application Ser. No. 10/678,562 claims compounds of Formula I, below, wherein R₃ may be selected from various groups including alkyl substituted with heteroaryl and heterocyclyl groups. Several series of these compounds, wherein R₂ was phenyl and R₄ was amino, were tested for hERG activity as described below.

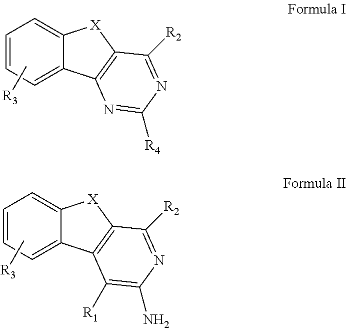

The R₃ groups may be substituted on position 6, 7, 8 or 9 on the included aromatic ring.

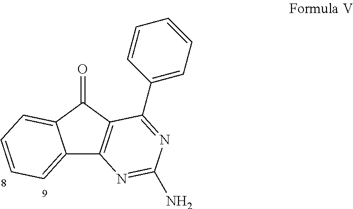

The unexpected discovery was made that a subset of compounds of Formula I, wherein R₂ was phenyl and R₄ was amino, with a variety of R₃ substituents in the 8 position had dramatically lower hERG activity as compared, for example, to compounds of Formula I, wherein R₂ was phenyl and R₄ was amino, with identical R₃ substituents in the 9 position. For Example

A compound of Formula V with a

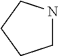

group in the 9 position has hERG binding of IC₅₀=4.2 μM while a compound with the same group in the 8 position has a hERG binding of only 1.4% @ 10 μM.

A compound of Formula V with a

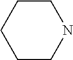

group in the 9 position has a hERG binding of IC₅₀=6.7 μM while a compound with the same group in the 8 position has a hERG binding of 42.7% @ 10 μM.

This lower hERG activity would be expected to result in compounds that have dramatically lower cardiac toxicity but retain excellent activity adenosine A1 and A2a receptor dual antagonists.

Shown below in Table 1 are the results of testing hERG activity and adenosine receptor activity in a series of compounds of Formula I wherein the R₃ substituent is in the 8 position. In Table 2 are the results of testing hERG activity and adenosine receptor in a series of compounds of Formula I wherein the R3 substituent is in the 9 position. As can be seen moving the substituent from the 9 to the 8 position results in dramatic decrease in hERG channel activity while retaining adenosine receptor activity. Thus compounds of Formula I with the R₃ substituent in the 8 position would be expected to be markedly superior with regard to cardiac toxicity especially the tendency to cause cardiac arrhythmias and sudden cardiac death.

Therefore, one aspect of the present invention features certain adenosine A1, A2a receptor dual antagonists. Specifically, the present invention provides compounds of Formula (I) below;

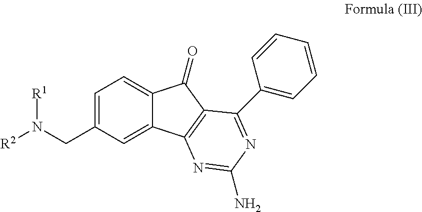

wherein
R¹ and R² are independently selected from H, optionally substituted C₁₋₃alkyl, and optionally substituted C₃₋₁₀cycloalkyl; or
R¹ and R² and the N atom they are attached to together form optionally substituted heterocyclyl having 0-2 additional heteroatoms selected from O, S, and N;
   or an optical isomer, enantiomer, diastereomer, racemate, or pharmaceutically acceptable salt thereof.

The compounds of Formula (I) are potent antagonists of adenosine A1 and adenosine A2a receptors. In addition, these compounds have surprisingly and unexpectedly discovered to have markedly reduced hERG channel binding effects. Accordingly, the compounds of Formula (I) are useful for the treatment of A1 and/or A2a receptor-mediated conditions while minimizing cardiac toxicity. Specifically, the invention provides compounds, compositions and methods to treat a condition that can be ameliorated by antagonizing Adenosine A1 and/or A2a receptors, which condition includes, but is not limited to, Parkinson's Disease, Huntington's Disease, Multiple System Atrophy, Corticobasal Degeneration, Alzheimer's Disease, and Senile Dementia. Furthermore, the compounds, compositions and methods of the present invention are particularly beneficial to a subject who has a condition selected from the group consisting of cancer, cardiac disease, neurological disease, neuro-endocrinological disease, and gastro-intestinal disease. More specifically, such subject has a condition selected from the group consisting of cardiac arrhythmia, colorectal cancer, episodic ataxia, and epilepsy.

In particular, R¹ and R² are independently selected from H and C₁₋₃alkyl optionally substituted with heterocyclyl. More specifically, the optional heterocyclyl substituent on said C₁₋₃alkyl is selected from

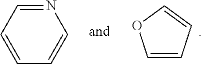

In particular, R¹ and R² and the N atom they are attached to together form optionally substituted heterocyclyl having 0-2 additional heteroatoms selected from O, S, and N. More specifically, the heterocyclyl formed by R¹, R² and the N atom they are attached to has one oxygen atom. Particularly, R¹ and R² and the N atom they are attached to together form optionally substituted

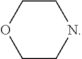

More specifically, the heterocyclyl formed by R¹, R² and the N atom they are attached to has a total of one nitrogen atom. Particularly, R¹ and R² and the N atom they are attached to together form optionally substituted heterocyclyl selected from

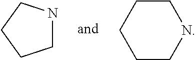

In particular, R¹ and R² and the N atom they are attached to together form an unsubstituted heterocyclyl selected from

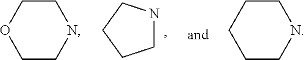

In particular, the present invention provides a compound of Formula (I)

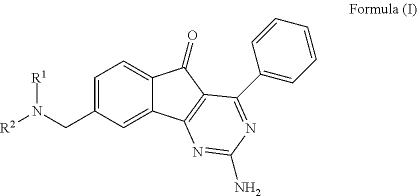

wherein
R¹ and R² are independently selected from H and methyl substituted with

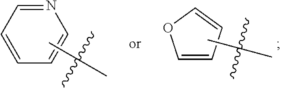

or
R¹ and R² and the N atom they are attached to together form a group selected from

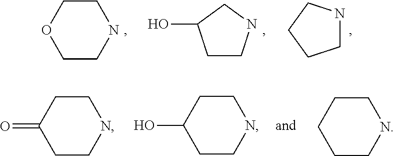

Particularly, R¹ and R² are independently selected from H and methyl substituted with

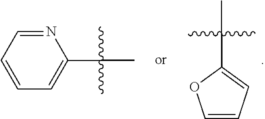

In particular, the present invention provides a compound of Formula (I) selected from:

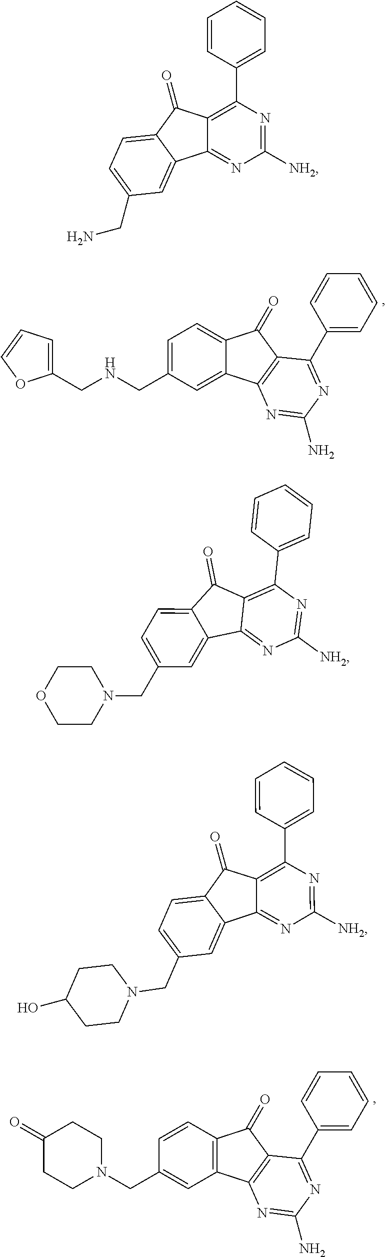

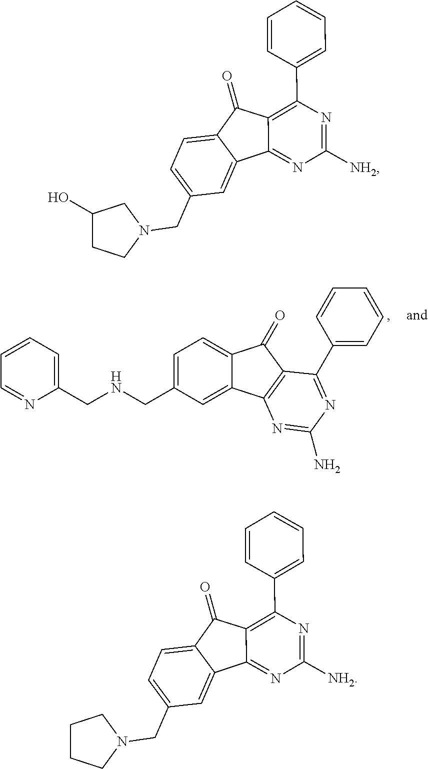

In particular, R¹ is H and R² is optionally substituted C₃₋₈cycloalkyl.

Another aspect of the present invention features a pharmaceutical composition comprising the compound of claim 1 and a pharmaceutically acceptable carrier.

Yet another aspect of the present invention features a method of treating a subject having a condition ameliorated by antagonizing adenosine A2a or A1 receptors in appropriate cells in the subject while minimizing cardiac toxicity, which comprises administering to the subject a therapeutically effective dose of a compound of Formula (I). Particularly, the condition is a neurodegenerative disorder or a movement disorder.

More particularly, the condition is selected from the group consisting of Parkinson's Disease, Huntington's Disease, Multiple System Atrophy, Corticobasal Degeneration, Alzheimer's Disease, and Senile Dementia.

In addition, the condition may be selected from the group consisting of cancer, cardiac disease, neurological disease, neuro-endocrinological disease, and gastro-intestinal disease. Specifically, the subject may have a condition selected from the group consisting of cardiac arrhythmia, colorectal cancer, episodic ataxia, and epilepsy.

Yet another aspect of the present invention features a method of preventing a condition ameliorated by antagonizing adenosine A2a or A1 receptors in the subject, comprising administering to the subject a prophylactically effective dose of a compound of Formula (I) either preceding or subsequent to an event anticipated to cause a condition ameliorated by antagonizing adenosine A2a or A1 receptors in appropriate cells in the subject. Particularly, the condition is a neurodegenerative disorder or a movement disorder. More particularly, the condition is selected from the group consisting of Parkinson's Disease, Huntington's Disease, Multiple System Atrophy, Corticobasal Degeneration, Alzheimer's Disease, and Senile Dementia. Further, the subject has a condition selected from the group consisting of cancer, cardiac disease, neurological disease, neuro-endocrinological disease, and gastro-intestinal disease. Specifically, the subject has a condition selected from the group consisting of cardiac arrhythmia, colorectal cancer, episodic ataxia, and epilepsy.

Yet another aspect of the present invention features a method of treating a subject having a condition ameliorated by antagonizing adenosine A2a and A1 receptors in appropriate cells in the subject, which comprises administering to the subject a therapeutically effective dose of a compound of Formula (I). Particularly, the condition is a neurodegenerative disorder or a movement disorder. More particularly, the condition is selected from the group consisting of Parkinson's Disease, Huntington's Disease, Multiple System Atrophy, Corticobasal Degeneration, Alzheimer's Disease, and Senile Dementia. Further, the subject has a condition selected from the group consisting of cancer, cardiac disease, neurological disease, neuro-endocrinological disease, and gastro-intestinal disease. Specifically, the subject has a condition selected from the group consisting of cardiac arrhythmia, colorectal cancer, episodic ataxia, and epilepsy.

Yet another aspect of the present invention features a method of preventing a condition ameliorated by antagonizing adenosine A2a and A1 receptors in the subject, comprising administering to the subject a prophylactically effective dose of a compound of Formula (I) either preceding or subsequent to an event anticipated to cause a condition ameliorated by antagonizing adenosine A2a or A1 receptors in appropriate cells in the subject. Particularly, the condition is a neurodegenerative disorder or a movement disorder. More particularly, the condition is selected from the group consisting of Parkinson's Disease, Huntington's Disease, Multiple System Atrophy, Corticobasal Degeneration, Alzheimer's Disease, and Senile Dementia. Further, the subject has a condition selected from the group consisting of cancer, cardiac disease, neurological disease, neuro-endocrinological disease, and gastro-intestinal disease. Specifically, the subject has a condition selected from the group consisting of cardiac arrhythmia, colorectal cancer, episodic ataxia, and epilepsy.

Yet another aspect of the present invention features a method of treating a subject having a condition ameliorated by antagonizing adenosine A2a and A1 receptors with reduced hERG channel binding in appropriate cells in the subject, which comprises administering to the subject a therapeutically effective dose of a compound of Formula (I). Particularly, the condition is a neurodegenerative disorder or a movement disorder. More particularly, the condition is selected from the group consisting of Parkinson's Disease, Huntington's Disease, Multiple System Atrophy, Corticobasal Degeneration, Alzheimer's Disease, and Senile Dementia. Further, the subject has a condition selected from the group consisting of cancer, cardiac disease, neurological disease, neuro-endocrinological disease, and gastro-intestinal disease. Specifically, the subject has a condition selected from the group consisting of cardiac arrhythmia, colorectal cancer, episodic ataxia, and epilepsy.

Yet another aspect of the present invention features a method of preventing a condition ameliorated by antagonizing adenosine A2a and A1 receptors with reduced hERG channel binding in the subject, comprising administering to the subject a prophylactically effective dose of a compound of Formula (I) either preceding or subsequent to an event anticipated to cause a condition ameliorated by antagonizing adenosine A2a or A1 receptors in appropriate cells in the subject. Particularly, the condition is a neurodegenerative disorder or a movement disorder. More particularly, the condition is selected from the group consisting of Parkinson's Disease, Huntington's Disease, Multiple System Atrophy, Corticobasal Degeneration, Alzheimer's Disease, and Senile Dementia. Further, the subject has a condition selected from the group consisting of cancer, cardiac disease, neurological disease, neuro-endocrinological disease, and gastro-intestinal disease. Specifically, the subject has a condition selected from the group consisting of cardiac arrhythmia, colorectal cancer, episodic ataxia, and epilepsy.

### DEFINITIONS

Unless otherwise noted, under standard nomenclature used throughout this disclosure the terminal portion of the designated side chain is described first, followed by the adjacent functionality toward the point of attachment.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

As used herein, the following chemical terms shall have the meanings as set forth in the following paragraphs:

The term “independently,” when in reference to chemical substituents, shall mean that when more than one substituent exists, the substituents may be the same or different.

Designated numbers of carbon atoms (e.g., C₁₋₈) shall refer independently to the number of carbon atoms in an alkyl or cycloalkyl moiety or to the alkyl portion of a larger substituent in which alkyl appears as its prefix root.

Unless specified otherwise, it is intended that the definition of any substituent or variable at a particular location in a molecule be independent of its definitions elsewhere in that molecule. It is understood that substituents and substitution patterns on the compounds of this invention can be selected by one of ordinary skill in the art to provide compounds that are chemically stable and that can be readily synthesized by techniques known in the art as well as those methods set forth herein.

Unless otherwise noted, when a particular group is “substituted” (e.g., alkyl, phenyl, aryl, heteroalkyl, heteroaryl), that group may have one or more substituents, preferably from one to five substituents, more preferably from one to three substituents, most preferably from one to two substituents, independently selected from the list of substituents.

As used herein, the term “alkyl,” whether used alone or as part of a substituent group, include straight and branched chains. For example, alkyl radicals include methyl, ethyl, propyl, isopropyl, butyl, isobutyl, sec-butyl, t-butyl, pentyl and the like. Unless otherwise noted, “C₁₋₄alkyl” means a carbon chain composition of 1-4 carbon atoms. Unless otherwise stated, the alkyl group will contain 1-20 carbon atoms. Unless otherwise stated, the alkyl group may be optionally substituted with one or more groups such as halogen, OH, CN, mercapto, nitro, amino, C₁-C₈-alkyl, C₁-C₈-alkoxyl, C₁-C₈-alkylthio, C₁-C₈-alkyl-amino, di(C₁-C₈-alkyl)amino, (mono-, di-, tri-, and per-) halo-alkyl, formyl, carboxy, alkoxycarbonyl, C₁-C₈-alkyl-CO—O—, C₁-C₈-alkyl-CO—NH—, carboxamide, hydroxamic acid, sulfonamide, sulfonyl, thiol, aryl, aryl(c₁-c₈)alkyl, heterocyclyl, and heteroaryl.

“Alkoxy” or “alkoxyl” means —O-alkyl and unless otherwise stated, it will have 1-8 carbon atoms.

“Halogen” means fluorine, chlorine, bromine or iodine

“Ac” means acyl.

“Aryl” or “Ar,” whether used alone or as part of a substituent group, is a carbocyclic aromatic radical including, but not limited to, phenyl, 1- or 2-naphthyl and the like. The carbocyclic aromatic radical may be substituted by independent replacement of 1 to 5 of the hydrogen atoms thereon with halogen, OH, CN, mercapto, nitro, amino, C₁-C₈-alkyl, C₁-C₈-alkoxyl, C₁-C₈-alkylthio, C₁-C₈-alkyl-amino, di(C₁-C₈-alkyl)amino, (mono-, di-, tri-, and per-) halo-alkyl, formyl, carboxy, alkoxycarbonyl, C₁-C₈-alkyl-CO—O—, C₁-C₈-alkyl-CO—NH—, or carboxamide. Illustrative aryl radicals include, for example, phenyl, naphthyl, biphenyl, fluorophenyl, difluorophenyl, benzyl, benzoyloxyphenyl, carboethoxyphenyl, acetylphenyl, ethoxyphenyl, phenoxyphenyl, hydroxyphenyl, carboxyphenyl, trifluoromethylphenyl, methoxyethylphenyl, acetamidophenyl, tolyl, xylyl, dimethylcarbamylphenyl and the like. “Ph” or “PH” denotes phenyl. “Bn” means benzyl.

The terms “heterocyclyl,” “heterocycle,” “heterocyclic,” and “heterocyclo” refer to an optionally substituted, unsaturated or partially saturated or fully saturated cyclic group which is, for example, a 4- to 7-membered monocyclic, 7- to 11-membered bicyclic, or 10- to 15-membered tricyclic ring system, which has at least one heteroatom in at least one carbon atom containing ring. Each ring of the heterocyclic group containing a heteroatom may have 1, 2, or 3 heteroatoms selected from nitrogen atoms, oxygen atoms, and sulfur atoms, where the nitrogen and sulfur heteroatoms may also optionally be oxidized. The nitrogen atoms may optionally be quaternized. The heterocyclic group may be attached at any heteroatom or carbon atom.

Exemplary heterocyclic groups include pyrrolidinyl; oxetanyl; pyrazolinyl; imidazolinyl; imidazolidinyl; oxazolyl; oxazolidinyl; isoxazolinyl; thiazolidinyl; isothiazolidinyl; tetrahydrofuryl; piperidinyl; piperazinyl; 2-oxopiperazinyl; 2-oxopiperidinyl; 2-oxopyrrolidinyl; 4-piperidonyl; tetrahydropyranyl; tetrahydrothiopyranyl; tetrahydrothiopyranyl sulfone; morpholinyl; thiomorpholinyl; thiomorpholinyl sulfoxide; thiomorpholinyl sulfone; 1,3-dioxolane; dioxanyl; thietanyl; thiiranyl; quinuclidinyl; tetrahydroisoquinolinyl; dihydroisoindolyl; dihydroquinazolinyl (such as 3,4-dihydro-4-oxo-quinazolinyl); dihydrobenzofuryl; dihydrobenzothienyl; dihydrobenzothiopyranyl; dihydrobenzothiopyranyl sulfone; dihydrobenzopyranyl; indolinyl; isochromanyl; isoindolinyl; piperonyl; tetrahydroquinolinyl; pyridinyl, pyrazinyl, pyrimidinyl, pyridazinyl, pyrroyl, pyrazolyl, imidazolyl, thiazolyl, oxazolyl, isoxazolyl, thiadiazolyl, triazolyl, triazinyl, oxadiazolyl, thienyl, furanyl, quinolinyl, isoquinolinyl, indolyl, isothiazolyl, 2-oxazepinyl, azepinyl, N-oxo-pyridyl, 1-dioxothienyl, benzothiazolyl, benzoxazolyl, benzothienyl, quinolinyl-N-oxide, benzimidazolyl, benzopyranyl, benzisothiazolyl, benzisoxazolyl, benzodiazinyl, benzofurazanyl, benzothiopyranyl, indazolyl, indolizinyl, benzofuryl, chromonyl, coumarinyl, cinnolinyl, quinoxalinyl, indazolyl, pyrrolopyridinyl, furopyridinyl (such as furo[2,3-c]pyridinyl, furo[3,2-b]pyridinyl, or furo[2,3-b]pyridinyl), imidazopyridinyl (such as imidazo[4,5-b]pyridinyl or imidazo[4,5-c]pyridinyl), naphthyridinyl, phthalazinyl, purinyl, pyridopyridyl, quinazolinyl, thienofuryl, thienopyridyl, thienothienyl, furyl and the like.

Substituted heterocyclyl may be further substituted with aryl, substituted aryl, heterocyclyl, or a second substituted heterocyclyl to give, for example, a 4-pyrazol-1-yl-phenyl or 4-pyridin-2-yl-phenyl.

Where the compounds according to this invention have at least one stereogenic center, they may accordingly exist as enantiomers. Where the compounds possess two or more stereogenic centers, they may additionally exist as diastereomers. Furthermore, some of the crystalline forms for the compounds may exist as polymorphs and as such are intended to be included in the present invention. In addition, some of the compounds may form solvates with water (i.e., hydrates) or common organic solvents, and such solvates are also intended to be encompassed within the scope of this invention.

Some of the compounds of the present invention may have trans and cis isomers. In addition, where the processes for the preparation of the compounds according to the invention give rise to mixture of stereoisomers, these isomers may be separated by conventional techniques such as preparative chromatography. The compounds may be prepared as a single stereoisomer or in racemic form as a mixture of some possible stereoisomers. The non-racemic forms may be obtained by either synthesis or resolution. The compounds may, for example, be resolved into their components enantiomers by standard techniques, such as the formation of diastereomeric pairs by salt formation. The compounds may also be resolved by covalent linkage to a chiral auxiliary, followed by chromatographic separation and/or crystallographic separation, and removal of the chiral auxiliary. Alternatively, the compounds may be resolved using chiral chromatography.

The present invention includes within its scope prodrugs of the compounds of this invention. In general, such prodrugs will be functional derivatives of the compounds that are readily convertible in vivo into the required compound. Thus, in the methods of treatment of the present invention, the term “administering” shall encompass the treatment of the various disorders described with the compositions specifically disclosed or with a composition which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

As used herein, the term “composition” is intended to encompass a product comprising the specified ingredients in the specified amounts, as well as any product which results, directly or indirectly, from combinations of the specified ingredients in the specified amounts.

The terms “subject” or “patient” are used herein interchangeably and as used herein, refer to any animal or artificially modified animal having a disorder ameliorated by antagonizing adenosine A2a and/or A1 receptors. In a preferred embodiment, the subject is a human. More particularly, the subject is a human being who is the object of treatment, observation or experiment.

The term “treating” or “treatment” as used herein, refers to actions that cause any indicia of success in the prevention or amelioration of an injury, pathology, symptoms or condition, including any objective or subjective parameters such as abatement; remission; diminishing of symptoms or making the injury, pathology, or condition more tolerable to the patient; slowing in the rate of degeneration or decline; making the final point of degeneration less debilitating; or improving a subject's physical or mental well-being. Thus the term “treatment” or “to treat” is intended to include any action that improves, prevents, reverses, arrests, abates, or inhibits the pathological process of a condition that can be ameliorated by antagonizing Adenosine A1 and/or A2a receptors as defined and used herein. The treatment or amelioration of symptoms can be based on objective or subjective parameters; including the results of a physical examination, neurological examination, and/or psychiatric evaluations.

As used herein, the term “concomitant administration” or “combination administration” of a compound, therapeutic agent or known drug with a compound of the present invention means administration of the drug and the one or more compounds at such time that both the known drug and the compound will have a therapeutic effect. In some cases this therapeutic effect will be synergistic. Such concomitant administration can involve concurrent (i.e. at the same time), prior, or subsequent administration of the drug with respect to the administration of a compound of the present invention. A person of ordinary skill in the art would have no difficulty determining the appropriate timing, sequence and dosages of administration for particular drugs and compositions of the present invention.

Accordingly, the term “treating” or “treatment” includes the administration of the compounds or agents of the present invention to treat, prevent, reverse, arrest, abate, or inhibit a condition that can be ameliorated by antagonizing Adenosine A1 and/or A2a receptors. In some instances, treatment with the compounds of the present invention will prevent, inhibit, or arrest the progression of a neurodegenerative or movement disorder. Examples of conditions treatable by the instant pharmaceutical composition include, without limitation, Parkinson's Disease, Huntington's Disease, Multiple System Atrophy, Corticobasal Degeneration, Alzheimer's Disease, and Senile Dementia.

The term “therapeutic effect” as used herein, refers to the treatment, inhibition, abatement, reversal, or prevention of a condition that can be ameliorated by antagonizing Adenosine A1 and/or A2a receptors, the effects or symptoms of such condition, or side effects of such condition in a subject.

The terms “a therapeutically effective amount” or “a therapeutically effective dose” are used interchangeably and, as used herein, mean a sufficient amount or dose of one or more of the compounds or compositions of the invention to produce a therapeutic effect, as defined above, in a subject or patient in need of such; treatment, inhibition, abatement, reversal, or prevention of that can be ameliorated by antagonizing Adenosine A1 and/or A2a receptors, the effects or symptoms of such condition, or side effects of such condition in a subject. The range of doses required for these different therapeutic effects will differ according to the characteristics of the subject or patient and the precise nature of the condition being treated.

In one embodiment, the therapeutically and/or prophylactically effective dose is a dose sufficient to deliver from about 0.001 mg/kg of body weight to about 200 mg/kg of body weight of the instant pharmaceutical composition. In another embodiment, the therapeutically and/or prophylactically effective dose is a dose sufficient to deliver from about 0.05 mg/kg of body weight to about 50 mg/kg of body weight. More specifically, in one embodiment, oral doses range from about 0.05 mg/kg to about 100 mg/kg daily. In another embodiment, oral doses range from about 0.05 mg/kg to about 50 mg/kg daily, and in a further embodiment, from about 0.05 mg/kg to about 20 mg/kg daily. In yet another embodiment, infusion doses range from about 1.0 mg/kg/min to about 10 mg/kg/min of inhibitor, admixed with a pharmaceutical carrier over a period ranging from about several minutes to about several days. In a further embodiment, for topical administration, the instant compound can be combined with a pharmaceutical carrier at a drug/carrier ratio of from about 0.001 to about 0.1.

The term “pharmaceutical dosage form” as that term is used herein, shall refer to a form of one or more of the compounds or compositions of this invention along with pharmaceutically acceptable excipients to produce a formulation suitable for administration to a subject. The form may be adapted for administration by any appropriate route including, but not limited to; oral, both immediate and delayed release, intravenous (I.V.), transdermal, intramuscular, intraventricular or nasal and may comprise; tablets, pills, capsules, semisolids, powders, sustained release formulations, solutions, suspensions, emulsions, syrups, elixirs, aerosols, or any other appropriate compositions.

This invention will be better understood by reference to the Experimental Details which follow, but those skilled in the art will readily appreciate that these are only illustrative of the invention as described more fully in the claims which follow thereafter. Additionally, throughout this application, various publications are cited. The disclosure of these publications is hereby incorporated by reference into this application to describe more fully the state of the art to which this invention pertains.

### Dosage Regimens

The present invention provides methods of treating a condition that can be ameliorated by antagonizing Adenosine A1 and/or A2a receptors in a human subject or patient using the arylindenopyrimidine compounds or compositions of the invention. The dosage schedule and amounts effective for this use, i.e., the dosing or dosage regimen, will depend on a variety of factors including the precise nature of the condition, disease or injury, the patient's physical status, weight, age and the like. In calculating the dosage regimen for a patient, the mode of administration is also taken into account.

The pharmaceutical compounds and compositions of the invention may be administered, for example, at a dosage of from about 10-3000 mg/day in a 70 kg human, preferably from 45-2500 mg/day in a 70 kg human, more preferably from about 50-2000 mg/day in a 70 kg human, or even more preferably from about 55-1500 mg/day in a 70 kg human, or most preferably from about 60-1200 mg/day in a 70 kg human. These dosages, however, may be varied depending the individual characteristics and tolerances of the subject and the on the precise nature of the condition being treated.

Based on this disclosure, a person of ordinary skill in the art will be able, without undue experimentation, having regard to that skill, to determine a therapeutically effective dose or amount of a particular compound of the invention for treating various disorders in humans. (see, e.g., Lieberman, Pharmaceutical Dosage Forms (Vols. 1-3, 1992); Lloyd, 1999, The art, Science and Technology of Pharmaceutical Compounding; and Pickar, 1999, Dosage Calculations).

A therapeutically effective dose is also one in which any toxic or detrimental side effects of the active agent is outweighed in clinical terms by therapeutically beneficial effects. It is to be further noted that for each particular subject, specific dosage regimens should be evaluated and adjusted over time according to the individual need and professional judgment of the person administering or supervising the administration of the compounds. It is also expected that the compositions of this invention could be initiated at a low or moderate dose and then increased to a fully therapeutically effective dose and blood level over a period of time.

For treatment purposes, the compositions or compounds disclosed herein can be administered to the subject, for example, in a single bolus delivery, via continuous delivery over an extended time period, or in a repeated administration protocol (e.g., by an hourly, daily or weekly, repeated administration protocol). The pharmaceutical formulations of the present invention can be administered, for example, one or more times daily, 3 times per week, or weekly. In one embodiment of the present invention, the pharmaceutical formulations of the present invention are orally administered once or twice daily.

In this context, a therapeutically effective dosage of the biologically active agent(s) can include repeated doses within a prolonged treatment regimen that will yield clinically significant results to prevent, reverse, arrest, or inhibit the epileptogenesis. Determination of effective dosages in this context is typically based on animal model studies followed up by human clinical trials and is guided by determining effective dosages and administration protocols that significantly reduce the occurrence or severity of targeted exposure symptoms or conditions in the subject. Suitable models in this regard include, for example, murine, rat, porcine, feline, non-human primate, and other accepted animal model subjects known in the art. Alternatively, effective dosages can be determined using in vitro models (e.g., immunologic and histopathologic assays). Using such models, only ordinary calculations and adjustments are typically required to determine an appropriate concentration and dose to administer a therapeutically effective amount of the biologically active agent(s) (e.g., amounts that are intranasally effective, transdermally effective, intravenously effective, or intramuscularly effective to elicit a desired response).

In an exemplary embodiment of the present invention, unit dosage forms of the compounds are prepared for standard administration regimens. In this way, the composition can be subdivided readily into smaller doses at the physician's direction. For example, unit dosages can be made up in packeted powders, vials or ampoules and preferably in capsule or tablet form.

The active compound present in these unit dosage forms of the composition can be present in an amount of, for example, from about 10 mg to about 800 mg or preferably in unit dosage amounts of about 10, 25, 50, 100, 200 250, 400, 450, 500, and 600 mg of one or more of the active arylindenopyrimidine compounds of the invention, for single or multiple daily administration, according to the particular need of the patient.

### Pharmaceutical Compositions

The present invention comprises pharmaceutical compositions containing one or more compounds of Formula (I) with a pharmaceutically acceptable carrier.

Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed.

If desired, tablets may be sugar coated or enteric coated by standard techniques. For parenteral use, the carrier will usually comprise sterile water, through other ingredients, for example, for purposes such as aiding solubility or for preservation, may be included. Injectable suspensions may also be prepared, in which case appropriate liquid carriers, suspending agents and the like may be employed.

The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 10 mg to about 1000 mg of one or more compounds of the invention and preferably unit doses of from about 10 mg to about 800 mg and more preferably in unit doses of about; 10 mg., 25 mg., 50 mg, 100 mg, 250 mg, 400 mg, 450 mg, 500 mg and 600 mg.

The pharmaceutical compositions may be administered at a dosage, for example, of from about 10-3000 mg/day in a 70 kg human, preferably from 25-2500 mg/day in a 70 kg human, more preferably from about 50-2000 mg/day in a 70 kg human, or even more preferably from about 55-1500 mg/day in a 70 kg human, or most preferably from about 60-1200 mg/day in a 70 kg human. The dosages, however, may be varied depending upon the requirements of the patient, the severity of the condition being treated and the compound being employed.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

Preferably these compositions are in unit dosage forms from such as tablets, pills, capsules, powders, granules, sterile parenteral solutions or suspensions, metered aerosol or liquid sprays, drops, ampoules, auto injector devices or suppositories; for oral, parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing, for example, from about 25 mg to about 800 mg of the active ingredient of the present invention.

The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a compound to treat or prevent a given condition.

One skilled in the art will further recognize that human clinical trails including first-in-human, dose ranging and efficacy trials, in healthy patients and/or those suffering from a given disorder, may be completed according to methods well known in the clinical and medical arts.

In general, the arylindenopyrimidine compounds of the present invention can be administered as pharmaceutical compositions by any method known in the art for administering therapeutic drugs including oral, buccal, topical, systemic (e.g., transdermal, intranasal, or by suppository), or parenteral (e.g., intramuscular, subcutaneous, or intravenous injection.) Administration of the compounds directly to the nervous system can include, for example, administration to intracerebral, intraventricular, intacerebroventricular, intrathecal, intracisternal, intraspinal or peri-spinal routes of administration by delivery via intracranial or intravertebral needles or catheters with or without pump devices.

Compositions can take the form of tablets, pills, capsules, semisolids, powders, sustained release formulations, solutions, suspensions, emulsions, syrups, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, can be found in such standard references as Alfonso A R: Remington's Pharmaceutical Sciences, 17th ed., Mack Publishing Company, Easton Pa., 1985, the disclosure of which is incorporated herein by reference in its entirety and for all purposes. Suitable liquid carriers, especially for injectable solutions, include water, aqueous saline solution, aqueous dextrose solution, and glycols.

The arylindenopyrimidine compounds can be provided as aqueous suspensions. Aqueous suspensions of the invention can contain an arylindenopyrimidinecompound in admixture with excipients suitable for the manufacture of aqueous suspensions. Such excipients can include, for example, a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate).

The aqueous suspension can also contain one or more preservatives such as ethyl or n-propyl p-hydroxybenzoate, one or more coloring agents, one or more flavoring agents, and one or more sweetening agents, such as sucrose, aspartame or saccharin. Formulations can be adjusted for osmolarity.

Oil suspensions for use in the present methods can be formulated by suspending a carbamate compound in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these.

Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compound of choice, alone or in combination with other suitable components can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations of the present invention suitable for parenteral administration, such as, for example, by intraarticular (in the joints), intravenous, intramuscular, intradermal, intraperitoneal, intraventricular and subcutaneous routes, can include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter.

Where the compounds are sufficiently soluble they can be dissolved directly in normal saline with or without the use of suitable organic solvents, such as propylene glycol or polyethylene glycol. Dispersions of the finely divided compounds can be made-up in aqueous starch or sodium carboxymethyl cellulose solution, or in suitable oil, such as arachis oil. These formulations can be sterilized by conventional, well-known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The concentration of a carbamate compound in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally acceptable diluents or solvent, such as a solution of 1,3-butanediol.

These formulations can be presented in unit-dose or multi-dose sealed containers, such as ampoules and vials. Injection solutions and suspensions can be prepared from sterile powders, granules, and tablets of the kind previously described.

An arylindenopyrimidine compound suitable for use in the practice of this invention can be and is preferably administered orally. The amount of a compound of the present invention in the composition can vary widely depending on the type of composition, size of a unit dosage, kind of excipients, and other factors well known to those of ordinary skill in the art. In general, the final composition can comprise, for example, from 1.0% percent by weight (% w) to 90% w of the carbamate compound, preferably 10% w to 75% w, with the remainder being the excipient or excipients.

Pharmaceutical formulations for oral administration can be formulated using pharmaceutically acceptable carriers well known in the art in dosages suitable for oral administration. Such carriers enable the pharmaceutical formulations to be formulated in unit dosage forms as tablets, pills, powder, dragees, capsules, liquids, lozenges, gels, syrups, slurries, suspensions, etc. suitable for ingestion by the patient.

Formulations suitable for oral administration can consist of (a) liquid solutions, such as an effective amount of the packaged nucleic acid suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions.

Pharmaceutical preparations for oral use can be obtained through combination of the compounds of the present invention with a solid excipient, optionally grinding a resulting mixture, and processing the mixture of granules, after adding suitable additional compounds, if desired, to obtain tablets or dragee cores. Suitable solid excipients are carbohydrate or protein fillers and include, but are not limited to sugars, including lactose, sucrose, mannitol, or sorbitol; starch from corn, wheat, rice, potato, or other plants; cellulose such as methyl cellulose, hydroxymethyl cellulose, hydroxypropylmethyl-cellulose or sodium carboxymethylcellulose; and gums including arabic and tragacanth; as well as proteins such as gelatin and collagen.

If desired, disintegrating or solubilizing agents can be added, such as the cross-linked polyvinyl pyrrolidone, agar, alginic acid, or a salt thereof, such as sodium alginate. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers.

Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

The compounds of the present invention can also be administered in the form of suppositories for rectal administration of the drug. These formulations can be prepared by mixing the drug with a suitable non-irritating excipient that is solid at ordinary temperatures but liquid at the rectal temperatures and will therefore melt in the rectum to release the drug. Such materials are cocoa butter and polyethylene glycols.

The compounds of the present invention can also be administered by intranasal, intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

The compounds of the present invention can be delivered transdermally, by a topical route, formulated as applicator sticks, solutions, suspensions, emulsions, gels, creams, ointments, pastes, jellies, paints, powders, and aerosols.

Encapsulating materials can also be employed with the compounds of the present invention and the term “composition” can include the active ingredient in combination with an encapsulating material as a formulation, with or without other carriers. For example, the compounds of the present invention can also be delivered as microspheres for slow release in the body. In one embodiment, microspheres can be administered via intradermal injection of drug (e.g., mifepristone)-containing microspheres, which slowly release subcutaneously (see Rao, J. Biomater Sci. Polym. Ed. 7:623-645, 1995; as biodegradable and injectable gel formulations (see, e.g., Gao, Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months. Cachets can also be used in the delivery of the compounds of the present invention.

The compositions of this invention can be administered in a variety of oral dosage form adapted for slow or controlled release. For example, the composition can be placed in an insoluble capsule with a hole at one end and a fluid absorbing distensible composition within the capsule opposite the perforated end. After administration, the fluid absorbing composition absorbs water from the patient's GI tract and swells and forces the active drug out through the perforation at a known and controllable rate. Many other delayed release or controlled release dosage forms known in the art can also be used in conjunction with the methods and compositions of this invention.

In another embodiment, the compounds of the present invention can be delivered by the use of liposomes which fuse with the cellular membrane or are endocytosed, i.e., by employing ligands attached to the liposome that bind to surface membrane protein receptors of the cell resulting in endocytosis. By using liposomes, particularly where the liposome surface carries ligands specific for target cells, or are otherwise preferentially directed to a specific organ, one can focus the delivery of the carbamate compound into target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

In other cases, the preferred preparation can be a lyophilized powder which can contain, for example, any or all of the following: 1 mM-50 mM histidine, 0.1%-2% sucrose, 2%-7% mannitol, at a pH range of 4.5 to 5.5, that is combined with buffer prior to use.

The pharmaceutical formulations of the invention can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms.

Pharmaceutically acceptable salts and esters refer to salts and esters that are pharmaceutically acceptable and have the desired pharmacological properties. Such salts include salts that may be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g. ethanolamine, diethanolamine, triethanolamine, tromethamine, N methylglucamine, and the like.

Pharmaceutically acceptable salts can also include acid addition salts formed from the reaction of amine moieties in the parent compound with inorganic acids (e.g. hydrochloric and hydrobromic acids) and organic acids (e.g. acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds. When there are two acidic groups present, a pharmaceutically acceptable salt or ester may be a mono-acid-mono-salt or ester or a di-salt or ester; and similarly where there are more than two acidic groups present, some or all of such groups can be salified or esterified.

Compounds named in this invention can be present in unsalified or unesterified form, or in salified and/or esterified form, and the naming of such compounds is intended to include both the original (unsalified and unesterified) compound and its pharmaceutically acceptable salts and esters. The present invention includes pharmaceutically acceptable salt and ester forms of Formula (I).

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid.

Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include the following; acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids and bases which may be used in the preparation of pharmaceutically acceptable salts include the following: acids; including acetic acid, 2,2-dichlorolacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydrocy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hippuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid and undecylenic acid; and bases; including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, sodium hydroxide, triethanolamine, tromethamine and zinc hydroxide.

A pharmaceutical composition of the invention can optionally contain, in addition to an arylindenopyrimidine compound, at least one other therapeutic agent useful in the treatment of a disease or condition that can be ameliorated by antagonizing Adenosine A1 and/or A2a receptors.

Methods of formulating pharmaceutical compositions have been described in numerous publications such as Pharmaceutical Dosage Forms: Tablets. Second Edition. Revised and Expanded. Volumes 1-3, edited by Lieberman et al; Pharmaceutical Dosage Forms: Parenteral Medications. Volumes 1-2, edited by Avis et al; and Pharmaceutical Dosage Forms Disperse Systems. Volumes 1-2, edited by Lieberman et al; published by Marcel Dekker, Inc, the disclosure of which are herein incorporated by reference in their entireties and for all purposes.

The pharmaceutical compositions are generally formulated as sterile, substantially isotonic and in full compliance with all Good Manufacturing Practice (GMP) regulations of the U.S. Food and Drug Administration.

### Kits

After a pharmaceutical comprising an arylindenopyrimidine compound has been formulated in a suitable carrier, it can be placed in an appropriate container and labeled for treatment of one or more conditions that can be ameliorated by antagonizing Adenosine A1 and/or A2a receptors. Additionally, another pharmaceutical comprising at least one other therapeutic agent useful in the treatment of such condition, or another disorder or condition associated thereof, can be placed in the container as well and labeled for treatment of the indicated disease(s). Such labeling can include, for example, instructions concerning the amount, frequency and method of administration of each pharmaceutical.

Although the foregoing invention has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. The following Examples are set forth to aid in the understanding of the invention, and are not intended and should not be construed to limit in any way the invention set forth in the claims which follow thereafter.

### General Synthetic Schemes

Representative compounds of the present invention can be synthesized in accordance with the general synthetic methods described below as well as the illustrative examples that follow. The products of some schemes can be used as intermediates to produce more than one of the instant compounds. The choice of intermediates to be used to produce subsequent compounds of the present invention can be a matter of discretion that is well within the capabilities of those skilled in the art. Since the schemes are an illustration, the invention should not be construed as being limited by the chemical reactions and conditions expressed. The preparation of the various starting materials used in the schemes is well within the skill of persons versed in the art.

Abbreviations or acronyms useful herein include:
Boc (tert-butyloxycarbonyl)
BuLi (butyllithium)
DMAP (4-(dimethylamino)pyridine)
DMF (dimethylformamide)
DMPU (1,3-Dimethyl-3,4,5,6-tetrahydro-2(1H)-pyrimidinone)
DMSO (methyl sulfoxide)
EDCI (1-(3-Dimethylaminopropyl)-3-ethylcarbodiimide hydrochloride)
EtOAc (ethyl acetate)
LCMS (high pressure liquid chroatography with mass spectrometer)
LHMDS (lithium hexamethyl disilazide)
NaHMDS (sodium hexamethyl disilazide)
NaO^{t}Bu (sodium tert-butoxide)
NBS (N-Bromosuccinimide)
NMP (N-Methyl-2-Pyrrolidinone)
SPE (solid phase extraction)
TEMPO (2,2,6,6-tetramethyl-1-piperidinyloxy, free radical)
TFA (trifluoroacetic acid);
THF (tetrahydrofuran)
TLC (thin layer chromatography)

The synthesis of the compounds of the present invention can be accomplished following, for example, the process shown in Scheme 1, wherein R¹ and R² are as described above.

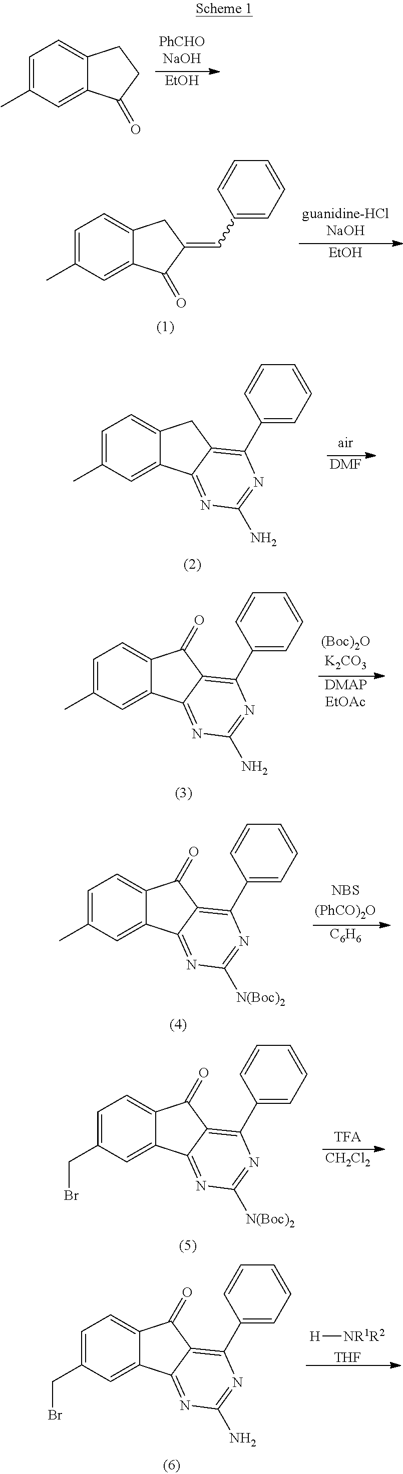

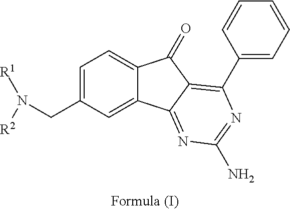

6-Methyl-1-indanone is reacted with benzaldehyde and sodium hydroxide to provide the enone (1) as a mixture of isomers. Treatment of enone (1) with guanidine forms the pyrimidine core. After oxidation of the methylene group to a ketone and protection of the aminopyrimidine, the aromatic methyl can be brominated with NBS in benzene. Deprotection is followed by displacement of the bromine with an amine (HNR¹R²) to yield the compounds of Formula (I). The final products are treated with HCl to yield the hydrochloride salts.

### EXAMPLES

The following experimental examples are provided, generally following Scheme I. However, some specific conditions were varied depending on the compound, such as reaction times, temperatures, and solvents used (for both reactions and purifications).

### Example 1

### Preparation of Enone (1)

To a mixture of 6-methyl-1-indanone (182.0 g, 1.25 mol) and benzaldehyde (90.0 mL, 0.89 mol) in 1.2 L of ethanol was added a solution of 62.0 g NaOH in 182.0 mL of H₂O. Additional ethanol (1 L) was added and the solution was stirred for 3 h at rt. The resulting precipitate was isolated by filtration and washed with cold EtOH. The residue was dried in a vacuum oven overnight at 60° C. to yield 197 g (95%) of enone (1) as a white solid.

### Example 2

### Preparation of Indenopyrimidine (3)

Guanidine-HCl (406 g, 4.25 mol) in ethanol (4 L) was neutralized by addition of solid NaOH (170 g, 4.25 mol) over 30 min. After a further 30 min, the solution was filtered and the residue washed with ethanol. The filtrate was then added to a solution of enone (1) (200 g, 0.85 mol) in 2 L of ethanol and the resulting mixture heated to 80° C. for 18 h. After cooling to 0° C., the suspension was filtered to yield 219 g of pyrimidine (2).

Pyrimidine (2) (219 g) was dissolved in 2 L of DMF and heated to 100° C. for 18 h while air was vigorously bubbled through the solution. The mixture was diluted with water and the resulting precipitate isolated by filtration. The resultant solid was dried in a vacuum oven for 18 h at 60° C. to yield 172 g (70%) of indenopyrimidine (3) as a yellow solid.

### Example 3

### Protection of Indenopyrimidine (3)

A solution of indenopyrimidine (3) (200.0 g, 0.70 mol), DMAP (8.5 g, 0.07 mol), K₂CO₃ (289.0 g, 2.10 mol), and (Boc)₂O (459.0 g, 2.10 mol) in 1.4 L of EtOAc was stirred at rt for 18 h. The reaction mixture was diluted with water (2 L) and EtOAc (3 L). The organic layer was washed with water (3×1 L). During the water washes a solid precipitated, which was isolated by filtration to yield 179 g of (4). The filtrate was evaporated to yield and additional 179 g of (4). The portions were combined and dried in a vacuum oven at 40° C. for 18 h to yield 275 g (81%) of (4) as a yellow solid.

### Example 4

### Bromination of Protected Pyrimidine (4)

A mixture of (4) (1.2 g, 2.5 mmol), N-bromosuccinimide (0.467 g, 2.6 mmol), and benzoyl peroxide (6 mg, 0.025 mmol) in 6 mL of benzene was heated to 90° C. for 16 h in a sealed tube. The solution was cooled to rt and then purified by column chromatography eluting with 5-10% EtOAc/hexanes to yield 813 mg (57%) of (5) as a yellow solid.

### Example 5

### Deprotection OF (5)

A solution of (5) (0.192 g, 0.34 mmol) and trifluoroacetic acid (1.2 g, 0.78 mL, 10.18 mmol) in 3 mL of CH₂Cl₂ was stirred at rt for 2 h. The solution was concentrated and neutralized by the addition of saturated aqueous NaHCO₃. The resulting mixture was filtered and the precipitate was dried to yield 115 mg (93%) of pyrimidine (6) as a yellow solid.

### Example 6

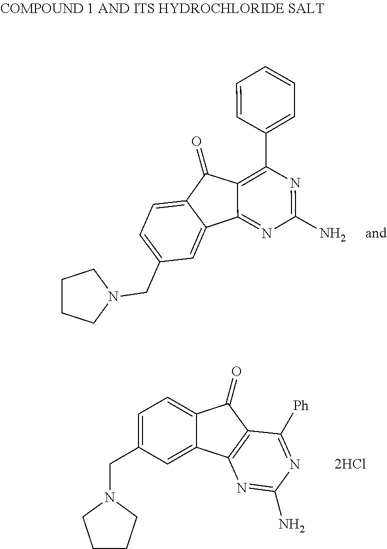

A solution of bromide (6) (79.9 g, 0.22 mol) and pyrrolidine (32 mL, 0.4 mol) in 1 L of THF was stirred at rt for 17 h. The reaction mixture was concentrated and diluted with CH₂Cl₂. The organic solution was washed with saturated Na₂CO₃ solution and then brine. The solution was then dried over Na₂SO₄, filtered and concentrated. The orange solid was purified by column chromatography eluting with 9:1:0.1 CH₂Cl₂:MeOH:NH₄OH to yield 54.22 g (70%) of Compound 1 as an orange solid.

To a solution of amine (Compound 1) (64.3 g, 0.18 mol) in 3.6 L of CH₂Cl₂ was added 5M HCl in isopropyl alcohol (180.0 mL, 0.2 mol) over 10 min. The reaction was stirred at rt for 2 h. The resulting precipitate was isolated by filtration, washed with CH₂Cl₂, and dried under high vacuum at 60° C. for two days to afford 56.80 g (72.4%) of the hydrochloride salt of Compound 1.

Following the general synthetic procedures outlined above and in Examples 1-6, the compounds of Table 1 below were prepared.

TABLE 1

| Compound | | Mass |
|---|---|---|
| No. | structure | Spec |
| (JNJ- 27631734) 1 | 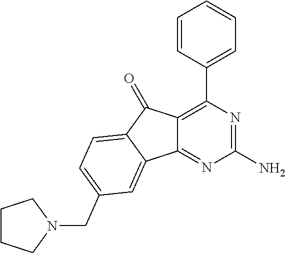 | 357 (M + 1) |
| (JNJ- 28849496) 2 | 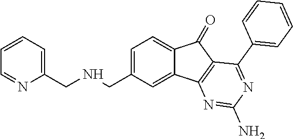 | 394 (M + 1) |
| (JNJ- 28648581) 3 | 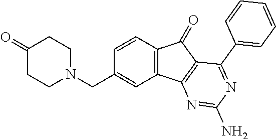 | 385 (M + 1) |
| (JNJ- 28644239) 4 | 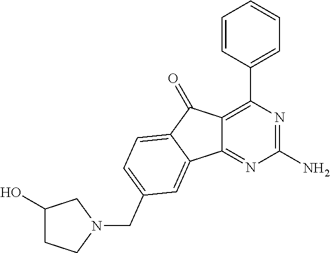 | 373 (M + 1) |
| (JNJ- 30998825) 5 | 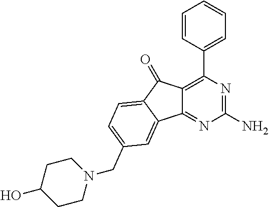 | 387 (M + 1) |
| (JNJ- 27666613) 6 | 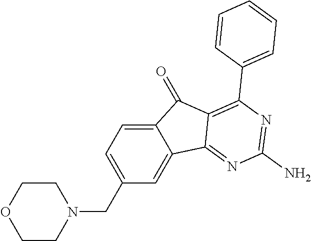 | 373 (M + 1) |
| (JNJ- 28845024) 7 | 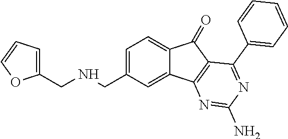 | 394 (M + 1) |

### Biological Assays and Activity

Ligand Binding Assay for Adenosine A2a Receptor

Ligand binding assay of adenosine A2a receptor was performed using plasma membrane of HEK293 cells containing human A2a adenosine receptor (PerkinElmer, RB-HA2a) and radioligand [³H]CGS21680 (PerkinElmer, NET1021). Assay was set up in 96-well polypropylene plate in total volume of 200 μl by sequentially adding 20 μL 1:20 diluted membrane, 130 μLassay buffer (50 mM Tris.HCl, pH7.4 10 mM MgCl₂, 1 mM EDTA) containing [³H] CGS21680, 50 μL diluted compound (4×) or vehicle control in assay buffer. Nonspecific binding was determined by 80 μM NECA. Reaction was carried out at room temperature for 2 hours before filtering through 96-well GF/C filter plate pre-soaked in 50 mM Tris.HCl, pH7.4 containing 0.3% polyethylenimine. Plates were then washed 5 times with cold 50 mM Tris.HCl, pH7.4, dried and sealed at the bottom. Microscintillation fluid 30 μl was added to each well and the top sealed. Plates were counted on Packard Topcount for [³H]. Data was analyzed in Microsoft Excel and GraphPad Prism programs. (Varani, K.; Gessi, S.; Dalpiaz, A.; Borea, P. A. British Journal of Pharmacology, 1996, 117, 1693; Tang, Y., Luo, J., Fleming, C. R., Kong Y., Olini, G. C., Wildey, M., Cavender, D. E., and Demarest, K. T. Assay and Drug Development Technologies, 2004, 2, 281.)

### Adenosine A2a Receptor Functional Assay

CHO-K1 cells overexpressing human adenosine A2a receptors and containing cAMP-inducible beta-galactosidase reporter gene were seeded at 40-50 K/well into 96-well tissue culture plates and cultured for two days. On assay day, cells were washed once with 200 L assay medium (F-12 nutrient mixture/0.1% BSA). For agonist assay, adenosine A2a receptor agonist NECA was subsequently added and cell incubated at 37° C., 5% CO₂ for 5 hrs before stopping reaction. In the case of antagonist assay, cells were incubated with antagonists for 5 minutes at R.T. followed by addition of 50 nM NECA. Cells were then incubated at 37° C., 5% CO₂ for 5 hrs before stopping experiments by washing cells with PBS twice. 50 μL 1× lysis buffer (Promega, 5× stock solution, needs to be diluted to 1× before use) was added to each well and plates frozen at −20° C. For β-galactosidase enzyme colorimetric assay, plates were thawed out at room temperature and 50 μL 2× assay buffer (Promega) added to each well. Color was allowed to develop at 37° C. for 1 h or until reasonable signal appeared. Reaction was then stopped with 150 μL 1M sodium carbonate. Plates were counted at 405 nm on Vmax Machine (Molecular Devices). Data was analyzed in Microsoft Excel and GraphPad Prism programs. (Chen, W. B.; Shields, T. S.; Cone, R. D. Analytical Biochemistry, 1995, 226, 349; Stiles, G. Journal of Biological Chemistry, 1992, 267, 6451)

### Adenosine A1 Functional Assay

CHOCREβgal#12/hA1-10 cells overexpressing human A1 receptors and containing cAMP-inducible β-galactosidase reporter gene were seeded at 50K/well in 96-well tissue culture plates and cultured for two days. On assay day, cells were washed with Assay Medium (F-12 nutrient mixture+0.1% BSA), followed by sequential addition of antagonist, agonist and forskolin. 50 nM R-PIA was used in antagonist assay. Forskolin was added last at 1 μM final concentration. Cells were then incubated at 37° C., 5% CO₂ for 5 hrs before stopping experiments by washing cells with PBS for 3 times. 50 μl lysis buffer (Promega) was added to each well and plates frozen at −20° C. For β-galactosidase enzyme colormetric assay, plates were thawed out at room temperature and 50 μl 2× assay buffer (Promega) added to each well. Color was allowed to develop at 37° C. for 1 hr. Reaction was then stopped with 150 μl M sodium carbonate. Plates were read at 405 nm on Vmax Machine (Molecular Devices). Data was analyzed in Microsoft Excel and GraphPad Prism programs. (Chen, W. B.; Shields, T. S.; Cone, R. D. Analytical Biochemistry, 1995, 226, 349; Stiles, G. Journal of Biological Chemistry, 1992, 267, 6451)

### hERG Channel Binding Assay

In the hERG channel binding assay, the ability of test compounds to displace ³H-astemizole (a high affinity hERG channel ligand) is measured.

The human ether-a-go-go related gene, hERG, expressed in the heart of human and other mammalian species encodes the pore-forming subunit of an ion channel called the rapidly activating delayed rectifier potassium current, Iₖᵣ. This ion channel is involved in repolarization of the cardiac action potential. Blocking the hERG K⁺ channels is an undesired property of pharmaceutics because inhibition of ion channels involved in cardiac repolarization can cause acquired long QT syndrome and may lead to a life-threatening form of ventricular arrhythmias called torsade de pointes. In susceptible individuals, syncope and sudden death can result from drug-induced block of this myocardial ion channel. Because the hERG K⁺ channel is a molecular target for QT prolongation, it is strategic to screen compounds for activity against this target at an early stage in drug discovery to de-risk development and reduce potential for undesirable side effects in humans.

A hERG binding assay is a primary screening method used to evaluate potential activity of a compound against the hERG potassium channel. The affinity of a compound for the hERG channel is measured in vitro by examining displacement of tritiated-[³H]astemizole, a high affinity hERG channel ligand. In this assay, membranes from HEK293 cells stably transfected with the human hERG channel cDNA are incubated with a range of concentrations of each test compound, vehicle (10% DMSO), or a positive control and [³H]astemizole. After a 1-hr incubation, the membranes are harvested onto GF/B filters, the filters dried and the radioactivity is measured.

For each test compound, percent inhibition of [³H]astemizole binding is calculated relative to control binding and IC₅₀ values determined by linear regression. Each assay includes a positive control (i.e., Cisapride) that is tested in parallel with test compounds.

At present, there is no gold standard preclinical test available to predict QT prolongation in humans. Therefore to assess potential cardiotoxicity, cardiovascular effects are evaluated preclinically in several different tests including the hERG channel binding assay.

Compounds listed in Table 2 below were tested in the above assays:

TABLE 2

| | Ki (nM) | | | |
|---|---|---|---|---|
| | | A2a | A1 | |
| Compound | A2a | antagonist | antagonist | |
| No. | binding | functional | functional | hERG binding |
| 1 | 6.69 | 3.08 | 24.4 | 1.4% at 10 μM |
| 2 | 10.2 | 5.43 | 26.29 | 34.5% at 10 μM |
| 3 | 9.4 | 5.9 | 73 | 13.8% at 10 μM |
| 4 | 7.9 | 4.3 | 57.6 | 27.8% at 10 μM |
| 5 | 5.8 | 2.5 | 91 | 26.2% at 10 μM |
| 6 | 5.39 | 1 | 1.8 | −12.5% at 10 μM |
| 7 | 10.8 | 5.33 | 12.5 | 9.0 μM (IC₅₀) |

The present invention is not to be limited in terms of the particular embodiments or examples described in this application, which are intended as single illustrations of individual aspects of the invention. Many modifications and variations of this invention can be made without departing from its spirit and scope, as will be apparent to those skilled in the art. Functionally equivalent methods and combinations within the scope of the invention, in addition to those enumerated herein will be apparent to those skilled in the art from the foregoing description, examples and accompanying drawings. Such modifications and variations are intended to fall within the scope of the appended claims. The present invention is to be limited only by the terms of the appended claims, along with the full scope of equivalents to which such claims are entitled.

### REFERENCES CITED

All references cited herein are incorporated herein by reference in their entirety and for all purposes to the same extent as if each individual publication or patent or patent application was specifically and individually indicated to be incorporated by reference in its entirety for all purposes.

The discussion of references herein is intended merely to summarize the assertions made by their authors and no admission is made that any reference constitutes prior art. Applicants reserve the right to challenge the accuracy and pertinence of the cited references.

What is claimed is:

1. A compound of Formula (I): 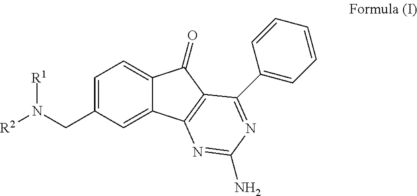
wherein
R¹ and R² are independently selected from H, optionally substituted C₁₋₃alkyl, and optionally substituted C₃₋₁₀cycloalkyl; or R¹ and R² and the N atom are attached together to form optionally substituted heterocyclyl having 0-2 additional heteroatoms selected from O, S, and N; or an optical isomer, enantiomer, diastereomer, racemate, or pharmaceutically acceptable salt thereof.

2. The compound of claim 1, wherein R¹ and R² are independently selected from H and C₁₋₃alkyl optionally substituted with heterocyclyl.

3. The compound of claim 2, wherein the optional heterocyclyl substituent on said C₁₋₃alkyl is selected from

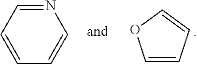

4. The compound of claim 1, wherein R¹ and R² and the N atom are attached together to form optionally substituted heterocyclyl having 0-2 additional heteroatoms selected from O, S, and N.

5. The compound of claim 4, wherein the heterocyclyl formed by R¹, R² and the N atom are attached to one O atom.

6. The compound of claim 5, wherein R¹ and R² and the N atom are attached together to form optionally substituted

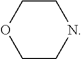

7. The compound of claim 4, wherein the heterocyclyl formed by R¹, R² and the N atom are attached to a total of one N atom.

8. The compound of claim 7, wherein R¹ and R² and the N atom are attached together to form optionally substituted heterocyclyl selected from

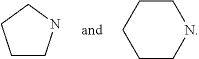

9. The compound of claim 5, wherein R¹ and R² and the N atom are attached together to form an unsubstituted heterocyclyl selected from

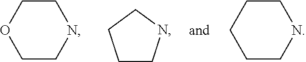

10. The compound of claim 1, wherein
R¹ and R² are independently selected from H and methyl substituted with 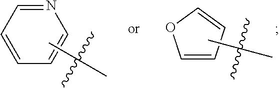
or
R¹ and R² and the N atom are attached together to form a group selected from

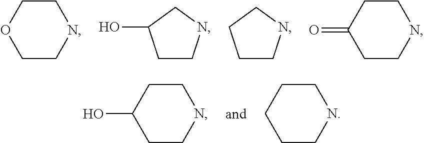

11. The compound of claim 1, wherein
R¹ and R² are independently selected from H and methyl substituted with 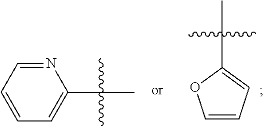
or
R¹ and R² and the N atom are attached together to form a group selected from

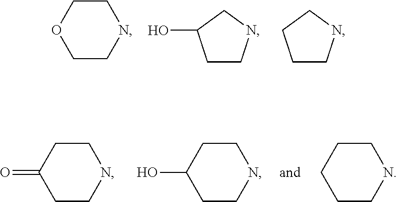

12. The compound of claim 1, which is

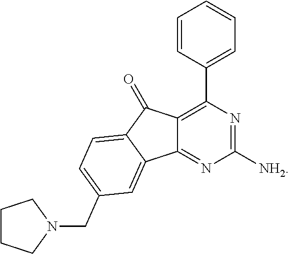

13. The compound of claim 1, which is.

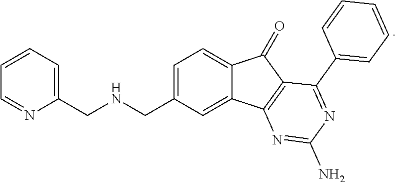

14. The compound of claim 1, which is.

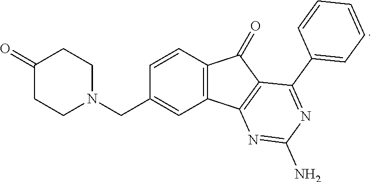

15. The compound of claim 1, which is

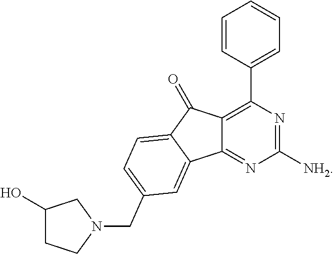

16. The compound of claim 1, which is

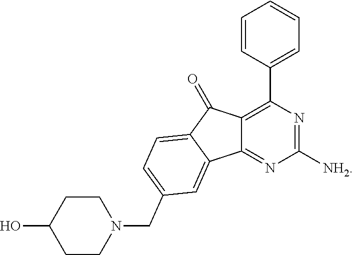

17. The compound of claim 1, which is

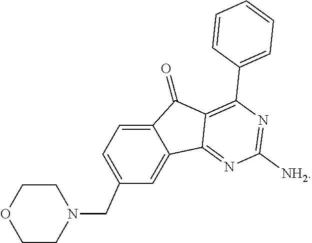

18. The compound of claim 1, which is

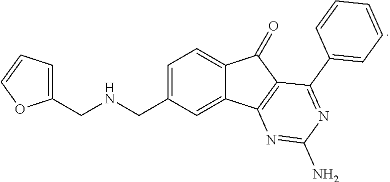

19. The compound of claim 1, which is

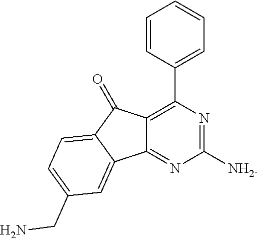

20. The compound of claim 1, wherein R¹ is H and R² is optionally substituted C₃₋₈cycloalkyl.

21. A pharmaceutical composition comprising the compound of claim 1 and a pharmaceutically acceptable carrier.

22. A method of treating a subject having a condition ameliorated by antagonizing adenosine A2a or A1 receptors in appropriate cells in the subject, which comprises administering to the subject a therapeutically effective dose of the compound of claim 1.

23. A method of preventing a condition ameliorated by antagonizing adenosine A2a or A1 receptors in the subject, comprising administering to the subject a prophylactically effective dose of the compound of claim 1 either preceding or subsequent to an event anticipated to cause a condition ameliorated by antagonizing adenosine A2a or A1 receptors in appropriate cells in the subject.

24. A method of treating a subject having a condition ameliorated by antagonizing adenosine A2a and A1 receptors in appropriate cells in the subject, which comprises administering to the subject a therapeutically effective dose of the compound of claim 1.

25. A method of preventing a condition ameliorated by antagonizing adenosine A2a and A1 receptors in the subject, comprising administering to the subject a prophylactically effective dose of the compound of claim 1 either preceding or subsequent to an event anticipated to cause a condition ameliorated by antagonizing adenosine A2a or A1 receptors in appropriate cells in the subject.

26. A method of treating a subject having a condition ameliorated by antagonizing adenosine A2a and A1 receptors with reduced hERG channel binding in appropriate cells in the subject, which comprises administering to the subject a therapeutically effective dose of the compound of claim 1.

27. A method of preventing a condition ameliorated by antagonizing adenosine A2a and A1 receptors with reduced hERG channel binding in the subject, comprising administering to the subject a prophylactically effective dose of the compound of claim 1 either preceding or subsequent to an event anticipated to cause a condition ameliorated by antagonizing adenosine A2a or A1 receptors in appropriate cells in the subject.

28. The method of claim 22, wherein the condition is a neurodegenerative disorder or a movement disorder.

29. The method of claim 22, wherein the condition is selected from the group consisting of Parkinson's Disease, Huntington's Disease, Multiple System Atrophy, Corticobasal Degeneration, Alzheimer's Disease, or Senile Dementia.

30. The method of claim 26, wherein the subject has a condition selected from the group consisting of cancer, cardiac disease, neurological disease, neuro-endocrinological disease, or gastro-intestinal disease.

31. The method of claim 30, wherein the subject has a condition selected from the group consisting of cardiac arrhythmia, colorectal cancer, episodic ataxia, or epilepsy.

32. The method of claim 22, wherein said compound (or enantiomer) or a pharmaceutically acceptable salt or ester thereof is administered in combination administration with one or more other compounds or therapeutic agents.

33. A kit comprising one or more therapeutically effective dosage forms of the pharmaceutical composition of claim 21.
